(19) United States
(12) Patent Application Publication
(10) Pub. No.: US 2025/0297044 A1
(43) Pub. Date: Sep. 25, 2025

(54) RADIATION-CURABLE COMPOSITION FOR USE IN A RAPID PROTOTYPING PROCESS OR A RAPID MANUFACTURING PROCESS

(71) Applicant: Kulzer GmbH, Hanau (DE)

(72) Inventors: Waltraut LOH, Heusenstamm (DE); Astrit KASTRATI, Wöllstadt (DE); Alfred HOHMANN, Schmitten (DE)

(73) Assignee: Kulzer GmbH, Hanau (DE)

(21) Appl. No.: 18/861,719

(22) PCT Filed: Apr. 28, 2023
(86) PCT No.: PCT/EP2023/061212

§ 371 Date: Oct. 30, 2024

(30) Foreign Application Priority Data
May 3, 2022 (DE) 10 2022 110 896.1

(51) Int. Cl. C08F 220/34; B33Y 70/00; B33Y 80/00; C08K 3/36

(52) U.S. Cl. CPC C08F 220/343; B33Y 70/00; B33Y 80/00; C08K 3/36

(57) ABSTRACT

Polymerizable, radiation-curable compositions are provided and comprise (i) monomers and (ii) at least one further component, wherein the (i) monomers comprise (a) at least one urethane (meth)acrylate that is at least difunctional, (b) at least one monofunctional acrylate having an alicyclic group and/or at least one monofunctional methacrylate having an alicyclic group, and (ii) the at least one further component comprises (c) at least one photoinitiator for the UV and/or visible range or a photoinitiator system for the UV and/or visible range. The compositions are usable for producing, via a rapid prototyping, manufacturing, or tooling process, dental prosthetic parts, orthopaedic instruments, or dental pre-forms that have a flexural strength of greater than or equal to 75 MPa, a modulus of elasticity of greater than or equal to 2600 MPa, and/or a water absorption of less than 45 [μg/mm3].

### FIELD OF INVENTION

The subject of the invention is a polymerizable, radiation-curable composition, in particular UV/Vis-, UV- or Vis-curable, comprising (i) monomers and (ii) at least one further component, wherein the (i) monomers comprise (a) at least one at least di-functional urethane (meth)acrylate (b) at least one monofunctional acrylate having an alicyclic group and/or at least one monofunctional methacrylate having an alicyclic group, and (ii) the at least one further component comprises (c) at least one photoinitiator for the UV and/or Vis range or a photoinitiator system for the UV and/or Vis range. The composition according to the invention is suitable for the production of blanks and three-dimensional molded bodies of dental prosthetic parts, orthopedic appliances, dental pre-forms, technical parts, tools, instruments, hoof repair parts or implants in medical prosthetics, which have a) a flexural strength of greater than or equal to 75 MPa and/or b) a modulus of elasticity of greater than or equal to 2600 MPa, and/or c) a water absorption of less than 45 [g/mm³], wherein these can be produced in a rapid prototyping or in a rapid manufacturing or rapid tooling process.

### BACKGROUND OF INVENTION

In addition to manual production methods, digital production methods are becoming increasingly important in the dental sector. Dental prostheses, such as crowns and bridges, have been produced subtractively using CAD-CAM technologies for several years. As only a small proportion of the material is used in this milling technology and the majority is discarded, and the tool can only work on one dental component at a time, it should be possible to achieve significant cost benefits in build-up procedures with the simultaneous production of many dental components and little or no waste.

Generative processes are already known in the dental sector, e.g. in the form of laser sintering from CoCr, Ti or polymers for the production of crowns and bridges, implant components or models.

Compositions of acrylates or derivatives of acrylates for the production of dental restorations with a corresponding property profile with regard to the mechanical requirements in the dental sector in accordance with DIN EN ISO 10477 are currently only available from a few manufacturers. Since only a few manufacturers have MPG Class IIa approval for their printable plastics and resins, and most resins can currently only be printed unfilled, the mechanical properties of currently available materials are always low. Therefore, these materials are not suitable for the fabrication of permanent restorations. A filled composition available on the market has the disadvantage of considerable sedimentation of the fillers and excessive water absorption according to ISO 10477:2020. If sedimentation is too high, a homogeneous printing result cannot be guaranteed with the long printing times. In addition, the user must first homogenize the composition quantitatively before use. There is therefore a need for compositions for the production of final prosthetic parts, orthopaedic appliances or dental pre-forms.

### BRIEF DESCRIPTION OF THE INVENTION

The problem of the invention was to provide a mixture of monomers, which can optionally comprise fillers, which after curing, in particular by means of radiation-curing processes, has good properties with regard to the modulus of elasticity, in particular a balance is to be achieved between the necessary strength while avoiding brittleness and at the same time a balance between a low viscosity as a requirement for printing while at the same time avoiding excessive sedimentation of the filler. In addition, the composition should be suitable for use in radiation-curing rapid manufacturing (RM) or rapid prototyping (RP) processes. A further problem was to provide a composition which contains inorganic fillers and has a high transparency, in particular as a polymerizable composition or as a polymerized composition. Furthermore, the problem was to specify a composition that has low water absorption, in particular in accordance with ISO 10477:2020, and is thus suitable for printing dental materials such as permanent dental prostheses.

In the present case, dental products are understood to mean, in particular, dental products that can be manufactured from polymerizable compositions, such as non-exhaustive full dentures, temporary crowns and bridges, inlays, onlays, full crowns, occlusal splints, drilling templates for implantology, splints for orthodontic corrections (similar to Invisalign), mouthguards, artificial teeth and brackets.

An object of the invention is a polymerizable, radiation-curable composition comprising (i) monomers and (ii) at least one further component, wherein (i) the monomers comprise
(a) at least one at least di-functional urethane (meth)acrylate,
(b) at least one monofunctional acrylate with an alicyclic group and/or at least one monofunctional methacrylate having an alicyclic group or mixtures thereof, in particular dicyclopentanylmethyl acrylate, and optionally
(d) at least one acrylic acid ester with an additional carboxy group, and (ii) comprising at least one further component
(c) at least one photoinitiator for the UV and/or Vis range or a photoinitiator system for UV and/or Vis range.

It is particularly preferred if (b) the at least one monofunctional acrylate with alicyclic group and/or the at least one monofunctional methacrylate with alicyclic group comprises at least one monofunctional acrylate with dicyclopentane group and/or at least one monofunctional methacrylate with dicyclopentane group or mixtures containing these. Preferably, the monofunctional acrylate with alicyclic group comprises at least one monofunctional acrylate with dicyclopentane group and/or at least one monofunctional methacrylate with dicyclopentane group, wherein the acrylate has from 14 to 25 C-atoms, in particular from 14 to 18 C-atoms. Dicyclopentanylmethyl acrylate is particularly preferred.

In a particularly preferred embodiment, the composition (d) comprises at least one acrylic acid ester with an additional carboxy group.

An alternative object of the invention is a polymerizable, radiation-curable composition comprising (i) monomers and (ii) at least one further component, wherein (i) the monomers comprise
(a) at least one at least di-functional urethane (meth)acrylate, and
(d) at least one acrylic acid ester with additional carboxy group, and (ii) which comprises at least one further component
(c) at least one photoinitiator for the UV and/or Vis range or a Photoinitiator system for UV and/or Vis range.

The compositions according to the invention are suitable for use in a generative printing process with layer-by-layer radiation-induced polymerization of the composition for the production of three-dimensional shaped bodies, in particular the composition has a viscosity of less than 7500 m·Pas, preferably less than 5000 m·Pas, in particular from 500 to less than 4000 m·Pas, preferably from 500 to 3000 m·Pas, particularly preferably from 1 to 1500 m-Pas. Further preferably, the composition has a viscosity of 100 to 1200 m·Pas.

The polymerization is preferably carried out by UV and/or Vis radiation-induced layer-by-layer polymerization of the composition to produce three-dimensional shaped bodies.

In one alternative, an object of the invention is a composition comprising (e) at least one disubstituted 4,4′-di(oxabenzole)dialkylmethane of formula I

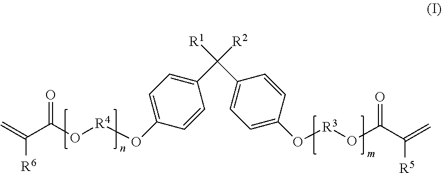

with R¹, R², R⁵ and R⁶ each independently selected from H or C1 to C4 alkyl, and with R³ and R⁴ each bivalently C1 to C4 alkylene, with n=0 to 6 and m=0 to 6, in particular n=1 and m=1 and/or n+m=2.

According to a particularly preferred alternative, the (d) at least one acrylic acid ester with additional carboxy group is selected from an acrylic acid ester with additional carboxy group of the formula II or III

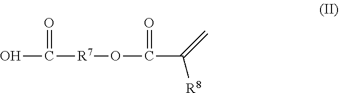

with R⁷ in each case independently selected from bivalent C, H, O and optionally N-containing groups having 1 to 25 C-atoms, in particular bivalent aromatic esters, aromatic urethanes, alkylene esters, alkyl urethanes, aromatic ethers, alkyl ethers, and R⁸ is selected from H and 1 to 4 C alkyl, preferably R⁷ is a bivalent aromatic ester,

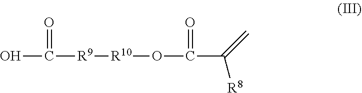

where R⁹ is independently selected from bivalent benzoyl, salicyloyl and derivatives thereof or —C—, R¹⁰ is bivalent —(OR)¹¹,—where R¹¹ is ethylene or propylene and where r is 0 to 10, in particular 1 to 6, preferably r=1 or R¹⁰ is independently selected from bivalent alkylene and R^{a} is selected from H and 1 to 4 C-alkyl, preferably R^{a} is H, methyl or ethyl.

Preferably, (d) the at least one acrylic acid ester with additional carboxy group comprises phthalic acid mono-[2-(methacryloyloxy)-ethyl ester], or 2-acryloyloxy ethylhydrogen phthalate, polyether-functionalized acrylic acid esters with carboxy group and mixtures thereof.

In an alternative, (b) the at least one monofunctional acrylate having an alicyclic group and/or at least one monofunctional methacrylate having an alicyclic group may comprise at least one monofunctional acrylate having a mono-valent alicyclic group and/or at least one monofunctional methacrylate having a mono-valent alicyclic group, in particular dicyclopentanylalkylene acrylate and/or dicyclopentanylalkylene methacrylate, in each case independently with alkylene comprising C1 to C6 atoms or mixtures containing at least one of the monomers.

Particularly preferred di-functional urethane (meth)acrylates having a bivalent alicyclic group comprise or are selected from bis-(4′,7′-dioxa-3′,8′-dioxo-2′-aza-decyl-9′-en)tetrahydro dicyclopentadiene, bis-(4′,7′-dioxa-3′,8′-dioxo-2′-aza-9′-methyl-decyl-9′-en)-tetrahydrodicyclo-pentadiene and/or mixtures thereof and optionally mixtures of the 3,8-/3,9-/4,8-/3,10-/4,10-isomers and/or the cis- and trans-isomers of the aforementioned compounds. Particularly preferred is the difunctional urethane acrylate with bivalent alicyclic group selected from bis-(4′,7′-dioxa-3′,8′-dioxo-2′-aza-decyl-9′-ene)tetrahydrodicyclopentadiene, bis-(4′,7′-dioxa-3′,8′-dioxo-2′-aza-9′-methyl-decyl-9′-ene)tetra-hydrodicyclopentadiene and/or mixtures thereof and optionally mixtures of the 3,8-/3,9-/4,8-/3,10-/4,10-isomers and/or the cis and trans isomers of the aforementioned compounds.

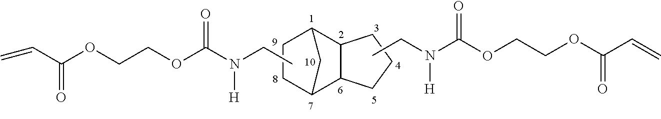

In a preferred embodiment, (a) the at least one di-functional urethane (meth)acrylate is selected from di-functional urethane (meth)acrylates having a bivalent alkylene group.

The di-functional urethane (meth)acrylate with bivalent alkylene group is preferably selected from linear or branched urethane dimethacrylates functionalized with a bivalent alkylene group, urethane dimethacrylate functionalized polyethers with alkylene group(s), such as bis(methacryloxy-2-ethoxycarbonylamino)alkylene, bis(methacryloxy-2-ethoxycarbonylamino) substituted polyalkylene ethers, preferably 1,6-bis(methacryloxy-2-ethoxycarbonylamino)-2,4,4-trimethylhexane, UDMA with alternative designation HEMA-TDMI. Preferred is a bis(methacryloxy-2-ethoxycarbonylamino)alkylene, wherein alkylene comprises linear or branched C3 to C20, preferably C3 to C6, as particularly preferably an alkylene substituted with methyl groups, such as HEMA-TMDI. The bivalent alkylene preferably comprises 2,2,4-trimethlyhexamethylene and/or 2,4,4-trimethylhexamethylene.

Furthermore, the composition may comprise at least one further monomer (g) at least one di-, tri-, tetra- or multi-functional monomer, which in particular is not a urethane (meth)acrylate and, in particular, does not correspond to formula I. The composition may also comprise a further mono-functional monomer (h).

In an alternative, (b) the at least one mono-functional acrylate with mono-valent alicyclic group and/or at least one mono-functional methacrylate with mono-valent alicyclic group may comprise tricyclodecane alkanol methacrylate and/or tricyclodecane alkanol acrylate with alkanol having 1 to 10 C-atoms or mixtures containing at least one of these monomers, preferred are tricyclodecane methanol acrylate, tricyclodecane methanol methacrylate, dicyclopentanyl acrylate, tricyclodecane ethanol acrylate, tricyclodecane ethanol methacrylate, isomers of the aforementioned monomers and/or mixtures thereof.

Likewise, a composition according to the invention preferably comprises as (e) at least one disubstituted 4,4′-di(oxabenzene)dialkylmethane of formula I

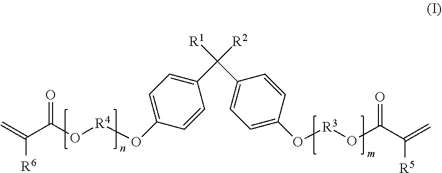

R¹ and R² each comprises methyl, and R⁵ and R⁶ being identical and selected from H, methyl and ethyl, in particular with R⁵ and R⁶ being identical and selected from H and methyl, and with R³ and R⁴ each independently bivalently ethylene or propylene with n=1 to 6, preferably n=2 to 4, and with m=1 to 6, preferably m=2 to 4, preferably with n=2 and m=2 or with n=4 and m=4 as well as mixtures thereof.

According to a further preferred alternative, the composition comprises as further components inorganic fillers selected from,
(f) inorganic fillers comprising inorganic oxides or inorganic mixed oxides and/or dental glasses, in particular zirconium dioxide, mixed oxides of a metal oxide and silicon dioxide, silicon dioxide, preferred fillers comprise silicon dioxide and/or mixed oxides with silicon dioxide. The inorganic fillers are particularly preferably silanized, especially silanized with acrylic-functional silanes, particularly preferably with (3-acryloyloxyalkyl)trimethoxysilanes with alkyl C1 to C6, preferably with (3-acryloyloxy propyl)trimethoxysilanes.

A particularly preferred composition comprising
(a) 1 to 90% by weight of at least one at least di-functional urethane (meth)acrylate,
(b) 1 to 50% by weight of at least one monofunctional acrylate having an alicyclic group and/or at least one monofunctional methacrylate containing an alicyclic group, and
(c) 0.01 to 10% by weight of at least one photoinitiator for the UV and/or Vis range or a photoinitiator system for the UV and/or Vis range,
(d) 1 to 50% by weight of at least one acrylic acid ester with additional carboxy group
(e) from 1 to 25% by weight of at least one disubstituted 4,4′-di(oxabenzene)dialkylmethane of formula I where R¹, R², R⁵ and R⁶ being each independently selected from H or C1 to C4-alkyl, and with R³ and R4 are each bivalently C1 to C4 alkylene, where n=0 to 6 and m=0 to 6, in particular n=1 and m=1 and/or n+m=2,
(f) 0 to 35% by weight of inorganic fillers comprising inorganic oxides or inorganic mixed oxides and/or dental glasses, in particular silicon dioxide, zirconium dioxide, mixed oxides with silicon dioxide, preferred fillers comprise silicon dioxide and/or mixed oxides of a metal oxide and silicon dioxide,
(g) 0 to 10% by weight, in particular 1 to 10% by weight of alkylene dimethacrylate and/or alkylene diacrylate, preferably with alkylene dimethacrylate, preferably in each case independently with alkylene C1 to C12 alkylene, preferably C1 to C4 alkylene, particularly preferably ethylene glycol dimethacrylate, and
(h) optionally 0.1 to 5% by weight, in particular 0.1 to 2% by weight of hydroxyethyl acrylate, the total composition being 100% by weight.

Further preferred is a composition comprising
(a) 30 to 70% by weight of at least one at least di-functional urethane (meth)acrylate,
(b) 10 to 40% by weight of at least one monofunctional acrylate with an alicyclic group and/or at least one monofunctional methacrylate with an alicyclic group,
(c) 0.01 to 10% by weight of at least one photoinitiator for the UV and/or Vis range or a photoinitiator system for the UV and/or Vis range,
(d) 5 to 40% by weight, in particular 5 to 25% by weight, of at least one acrylic acid ester with additional carboxy group,
(e) 1 to 15% by weight, in particular 2 to 5% by weight, of at least one disubstituted 4,4′-di(oxabenzole)dialkylmethane of the formula I where R¹, R², R⁵ and R⁶ are each independently selected from H or C1 to C4 alkyl, and with R³ and R⁴ each bivalently C1 to C4 alkylene, with n=0 to 6 and m=0 to 6, in particular n=1 and m=1 and/or n+m=2,
(f) 0 to 35% by weight of inorganic fillers comprising inorganic oxides or inorganic mixed oxides and/or dental glasses, in particular silicon dioxide, zirconium dioxide, mixed oxides with silicon dioxide and a further metal oxide, preferred fillers comprise silicon dioxide and/or mixed oxides with silicon dioxide and a further metal oxide,
(g) 0 to 10% by weight, in particular 1 to 10% by weight of alkylene dimethacrylate and/or alkylene diacrylate, preferably with alkylene dimethacrylate, preferably in each case independently with alkylene C1 to C12 alkylene, preferably C1 to C4 alkylene, particularly preferably ethylene glycol dimethacrylate,
(h) optionally 0.1 to 5% by weight, in particular 0.1 to 2% by weight of hydroxyethyl acrylate, the total composition being 100% by weight.

Further preferred is a composition comprising
(a) 40 to 60% by weight of at least one at least di-functional urethane (meth)acrylate,
(b) 15 to 30% by weight of at least one monofunctional acrylate with an alicyclic group and/or at least one monofunctional methacrylate containing an alicyclic group,
(c) 0.01 to 10% by weight of at least one photoinitiator for the UV and/or Vis range or a photoinitiator system for the UV and/or Vis range,
(d) 5 to 30% by weight, in particular 5 to 25% by weight, of at least one acrylic acid ester with additional carboxy group,
(e) 1 to 15% by weight, in particular 2 to 5% by weight, of at least one disubstituted 4,4′-di(oxabenzol)dialkylmethane of the formula I, where R¹, R², R⁵ and R⁶ are each independently selected from H or C1 to C4-alkyl, and with R³ and R⁴ each bivalently C1 to C4-alkylene, where n=0 to 6 and m=0 to 6, in particular n=1 and m=1 and/or n+m=2,
(f) 0 to 35% by weight of inorganic fillers comprising inorganic oxides or inorganic mixed oxides and/or dental glasses, in particular silicon dioxide, zirconium dioxide, mixed oxides with silicon dioxide, preferred fillers comprise silicon dioxide and/or mixed oxides with silicon dioxide and a further metal oxide,
(g) 0 to 10% by weight, in particular 1 to 10% by weight of alkylene dimethacrylate and/or alkylene diacrylate, preferably with alkylene dimethacrylate, preferably in each case independently with alkylene C1 to C12 alkylene, preferably C1 to C4 alkylene, particularly preferably ethylene glycol dimethacrylate,
   wherein the total composition is 100% by weight.

Optionally, additionally (h) 0.1 to 5% by weight, in particular 0.1 to 2% by weight of hydroxyethyl acrylate may be present in the composition.

A particularly preferred composition comprises
(a) 1 to 90% by weight of at least one at least di-functional urethane (meth)acrylate,
(b) 1 to 50% by weight, in particular 20 to 35% by weight, of at least one monofunctional acrylate with an alicyclic group and/or at least one monofunctional methacrylate with an alicyclic group, and
(c) 0.01 to 10% by weight of at least one photoinitiator for the UV and/or Vis range or a photoinitiator system for the UV and/or Vis range,
(d) optionally 1 to 10 wt. %, in particular 1 to 9 wt. %, preferably 1 to 7 wt. %, at least one acrylic acid ester with additional carboxy group,
(e) 1 to 25% by weight, in particular 2 to 5% by weight, of at least one disubstituted 4,4′-di(oxabenzol)dialkylmethane of the formula I, with R¹, R², R⁵ and R⁶ each independently selected from H or C1 to C4-alkyl, and with R³ and R⁴ each bivalently C1 to C4-alkylene, where n=0 to 6 and m=0 to 6,
(f) 0 to 35% by weight of inorganic fillers comprising inorganic oxides or inorganic mixed oxides and/or dental glasses, in particular zirconium dioxide, mixed oxides of with silicon dioxide, silicon dioxide, preferred fillers comprise silicon dioxide and/or mixed oxides with silicon dioxide and a metal oxide, the total composition being amounts to 100% by weight, optionally, additional may be present in the composition
(g) 0 to 10% by weight, in particular 1 to 10% by weight of alkylene dimethacrylate and/or alkylene diacrylate, preferably with alkylene dimethacrylate, preferably in each case independently with alkylene C1 to C12 alkylene, preferably C1 to C4 alkylene, particularly preferably ethylene glycol dimethacrylate, and/or optionally
(h) 0.1 to 5% by weight, in particular 0.1 to 2% by weight of hydroxyethyl acrylate.

Further preferred is a composition comprising
(a) 30 to 70% by weight, in particular 40 to 60% by weight, of at least one at least di-functional urethane (meth)acrylate,
(b) 10 to 40% by weight, in particular 15 to 35% by weight, of at least one monofunctional acrylate with an alicyclic group acrylate with an alicyclic group and/or at least one monofunctional methacrylate with an alicyclic group,
(c) 0.01 to 10% by weight of at least one photoinitiator for the UV and/or Vis range or a photoinitiator system for the UV and/or Vis range,
(d) optionally 1 to 9% by weight, in particular 1 to 7% by weight, of at least one acrylic acid ester with additional carboxy group,
(e) 1 to 15% by weight, in particular 2 to 5% by weight, of at least one disubstituted 4,4′-di(oxabenzol)dialkylmethane of the formula I, where R¹, R², R⁵ and R⁶ are each independently selected from H or C1 to C4-alkyl, and with R³ and R⁴ each bivalently C1 to C4-alkylene, where n=0 to 6 and m=0 to 6,
(f) 0 to 35% by weight of inorganic fillers comprising inorganic oxides or inorganic mixed oxides and/or dental glasses, in particular silicon dioxide, zirconium dioxide and/or mixed oxides with silicon dioxide and a further metal oxide, and
(g) 0 to 10% by weight, in particular 1 to 10% by weight of alkylene dimethacrylate and/or alkylene diacrylate, preferably with alkylene dimethacrylate, preferably in each case independently with alkylene C1 to C12 alkylene, preferably C1 to C4 alkylene, particularly preferably ethylene glycol dimethacrylate,
(h) optionally 0.1 to 5% by weight, in particular 0.1 to 2% by weight of hydroxyethyl acrylate,
   the total composition being 100% by weight.

Further preferred is a composition comprising
(a) 30 to 70% by weight, in particular 40 to 60% by weight, of at least one at least di-functional urethane (meth)acrylate,
(b) optionally 10 to 40% by weight, in particular 15 to 30% by weight, of at least one monofunctional acrylate with an alicyclic group and/or at least one monofunctional methacrylate with an alicyclic group,
(c) 0.01 to 10% by weight of at least one photoinitiator for the UV and/or Vis range or a photoinitiator system for the UV and/or Vis range,
(d) from 1 to 60% by weight, preferably from 5 to 60% by weight, particularly preferably from 10 to 50% by weight, in particular 10 to 40% by weight of at least one acrylic acid ester with additional carboxy group
(e) optionally 5 to 30% by weight, in particular 5 to 15% by weight, of at least one disubstituted 4,4′-di(oxabenzol)dialkylmethane of the formula I where R¹, R², R⁵ and R⁶ are each independently selected from H or C1 to C4-alkyl, and with R³ and R⁴ each bivalently C1 to C4-alkylene, where n=0 to 6 and m=0 to 6,
(f) 0 to 35% by weight, in particular 1 to 25% by weight, of inorganic fillers comprising inorganic oxides or inorganic mixed oxides and/or dental glasses, in particular zirconium dioxide, mixed oxides with silicon dioxide, silicon dioxide, preferred fillers comprise silicon dioxide and/or mixed oxides with silicon dioxide and a further metal oxide,
(g) 0 to 10% by weight, in particular 1 to 7% by weight of alkylene dimethacrylate and/or alkylene diacrylate, preferably with alkylene dimethacrylate, preferably in each case independently with alkylene C1 to C12 alkylene, preferably C1 to C4 alkylene, particularly preferably ethylene glycol dimethacrylate,
(h) optionally 0.1 to 5% by weight, in particular 0.1 to 2% by weight of hydroxyethyl acrylate,
the total composition being 100% by weight.

When selecting the monomers, it has to be paid attention to ensuring that they bond well with the optional filler. As a rule, polyurethanes, acrylates, polyesters and other monomers do not bond well with the fillers used. For this reason, the fillers are usually silanized or hydrophobized on the surfaces for improved bonding with the monomers. The fillers according to the invention are preferably silanized on the surface with silanes containing acrylate groups.

Surprisingly, it was found that acidic monomers also adhere well to the filler particles. According to the invention, therefore, a composition is provided which comprises a polymerizable monomer having a free carboxy group and/or an anhydride group, the composition additionally comprising a difunctional acrylate or methacrylate having an alicyclic group and at least one photoinitiator.

If, due to the specific dental application, no inorganic fillers can be used in the polymerizable composition, for example due to the desired viscosity of the composition, it is possible to use dyes or pigments in the composition to reflect the radiation, in particular diffuse reflection or scattering of the irradiated radiation. Dyes are compounds that are soluble in the polymerizable composition and preferably form a clear solution.

The radiation-curable compositions according to the invention can preferably be irradiated with a radiation source which emits light in the Vis range, particularly preferred are radiation sources which emit radiation from 360 to 750 nm, in particular at about 385 nm, particularly preferred at about 405 nm. Particularly preferably, the composition according to the invention can be irradiated with a polychromatic radiation source, such as a DLP projector, or preferably with a monochromatic radiation source, such as a laser projector, in the Vis range from 380 to 660 nm.

If these pigments and/or dyes are added, the photoinitiator content in the composition can be reduced. If the photoinitiator content is too high, this can lead to so-called “overcuring” of the irradiated composition, i.e. embrittlement, so that the dental parts produced accordingly cannot be used.

The use of the inorganic fillers, pigments or dyes according to the invention leads to a uniform scattering of the radiation sources, in particular the UV and Vis radiation source in the monomer matrix of the composition, so that a more uniform curing of the composition is assumed. As a result, the polymerized compositions exhibit increased values for the fracture work achieved.

The composition according to the invention has the following properties after exposure with a radiation source in the Vis range, in particular from 385 to 405 nm, preferably after exposure in a stereolithography process and obtaining a polymerized composition preferably in the form of a blank, dental prosthetic part, orthopaedic appliance or dental preform, as well as optional post-treatment of the polymerized composition with a radiation source, orthopaedic appliance or dental preform, as well as optional post-treatment of the polymerized composition with a radiation source, has the following properties a) a flexural strength of greater than or equal to 75 MPa and/or b) a modulus of elasticity of greater than or equal to 2600 MPa, in particular greater than or equal to 2700 MPa, in each case determined according to DIN EN ISO 10477:2020.

Post-curing or post-hardening can preferably be carried out using a laboratory light unit (HiLite Power 3D) or in a light oven, preferably with a light spectrum of 390-540 nm.

The subject of the invention is a polymerizable, radiation-curable composition which can be polymerized, in particular by means of UV/VIS, UV or VIS radiation, comprising (i) monomers, preferably a mixture of monomers, and (ii) at least one further component, wherein the (i) monomers comprise
(a) at least one at least di-functional urethane (meth)acrylate,
(b) at least one monofunctional acrylate having an alicyclic group and/or at least a monofunctional methacrylate with an alicyclic group, and/or
(d) at least one acrylic acid ester with additional carboxy group, and/or
(e) optionally at least one disubstituted 4,4′-di(oxabenzene)dialkylmethane of formula I wherein R¹, R², R⁵ and R⁶ are each independently selected from H or C1 to C4 alkyl, in particular with R¹ and R² equal to C1 to C4 alkyl, preferably methyl, and with R⁵ and R⁶ equal to and selected from H, methyl or ethyl, in particular with R⁵ and R⁶ equal to and selected from H or methyl, and with R³ and R⁴ each bivalently C1 to C4 alkylene, where n=2 to 6 and m=2 to 6, and (ii) at least one further component, (c) at least one photoinitiator for the UV and/or Vis range or a photoinitiator-system.

In preferred alternatives, in formula I R¹ and R² may each be methyl, and R⁵ and R⁶ may be the same and selected from H, methyl and ethyl, preferably R⁵ and R⁶ are the same and selected from H and methyl, and with R³ and R⁴ each independently bivalent ethylene or propylene with n=1 to 6, preferably n=2 to 4, and with m=1 to 6, particularly preferably n=2 to 4 and m=2 to 4, further preferably with n=2 and m=2 or with n=4 and m=4 as well as mixtures thereof. Particularly preferred is a mixture of 4,4′-di(oxabenzene)dialkylmethane of the formula I a) where R¹ and R² are each methyl, and R⁵ and R⁶ are each H and where R³ and R⁴ are each independently bivalent ethylene with n=1 to 6, preferably n=2 to 4, and with m=1 to 6, preferably m=2 to 4, preferably with n=4 and m=4 as well as mixtures thereof in mixtures with b) with R¹ and R² in each case methyl-, and R⁵ and R⁶ equal methyl and with R³ and R⁴ in each case independently bivalent ethylene with n=1 to 6, preferably n=1 to 4 and m=1 to 4, preferably with n=1 and m=1 as well as mixtures thereof.

Optionally, the composition may have a content of alkylene dimethacrylate and/or alkylene diacrylate, preferably each independently with alkylene C1 to C12 alkylene of 0 to 10% by weight, in particular from 0 to 5% by weight, preferably from 0.001 to 5% by weight, the total content of the composition being 100% by weight.

Optionally, the composition may additionally comprise at least one polyether diacrylate, such as poly(ethylene glycol) diacrylate, poly(ethylene glycol) di(alkyl) acrylate, poly(propylene glycol) diacrylate, poly(propylene glycol) di(alkyl) acrylate with alkyl having 1 to 10 C-atoms, preferably 1 to 4 C-atoms, or a mixture containing at least two of the said monomers, in particular each independently with at least 2 ethylene glycol or propylene glycol units, preferably 3 to 15. Preferred polyether diacrylates may be selected from triethylene glycol dimethacrylate, diethylene glycol dimethacrylate and/or tetraethylene glycol dimethacrylate. Alternatively, or additionally, the composition may comprise diacrylates selected from decanediol di(meth)acrylate, dodecanediol di(meth)acrylate, hexyldecanediol di(meth)-acrylate, butanediol di(meth)acrylate or mixtures containing at least one of the acrylates.

The designation in brackets in the terms (methyl) acrylate or (alkyl) acrylate means that the acrylates can be present as acrylate or methyl acrylate and alternatively as alkyl acrylate.

It is further preferred if the composition contains as (d) the at least one acrylic acid ester with additional carboxy group, acrylic acid ester with at least one additional anhydride group of carboxy groups and/or the at least one derivative of the aforementioned acrylic acid esters, in particular (alkyl)acrylic acid ester with alkyl C1 to C4 alkyl groups, preferably with alkyl equal to methyl or ethyl, selected from an acrylic acid ester with additional carboxy group of formula II or III,

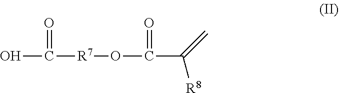

with R⁷ in each case independently selected from bivalent C, H, O-containing groups with 1 to 50 C-atoms, in particular with 1 to 25 C-atoms, preferably 8 to 25 C-atoms, in particular bivalent aromatic esters, alkylene esters, aromatic ethers, alkyl ethers, and R^{a} is selected from H and 1 to 4 C-alkyl, preferably R⁷ is a bivalent aromatic ester, preferably an ester of a phthalate and R⁸ is H, methyl or ethyl,

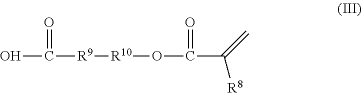

wherein R⁹ can be independently selected from bivalent benzoyl, salicyloyl or —C—, R¹⁰ can be a bivalent —(OR)¹¹ᵣ—, with R¹¹ equal to ethylene or propylene and with r=0 to 8, in particular with r=1 to 4, preferably r=1, particularly preferably —(OR¹¹)— can be a bivalent group derived from poly(alkylene glycol), in particular poly(propylene glycol) or poly(ethylene glycol) with 1 to 6 propylene glycol or ethylene glycol units, or R¹⁰ can be independently selected from bivalent alkylene and R^{a} can be selected from H and 1 to 4 C-alkyl, preferably R^{a} is H, methyl or ethyl.

Another object of the invention is a composition which can comprise as (d) at least one acrylic acid ester with additional carboxy group, acrylic acid ester with at least one additional anhydride group of carboxy groups and/or derivative of the aforementioned acrylic acid esters, in particular phthalic acid mono-[2-(methacryloyloxy)-ethyl ester] or 2-acryloyloxyethyl hydrogen phthalate, 2-(acryloyloxy)ethyl 2-hydroxyethyl phthalate, polyether-functionalized acrylic acid esters with carboxy group, preferably the composition comprises polyether-functionalized acrylic acid esters with carboxy group on acrylic, polyether-functionalized acrylic acid esters with carboxy group on alkyl, in particular the polyethers being based on poly(propylene glycol) and poly(ethylene glycol) with 1 to 6 glycol units.

According to the invention, the composition comprises 2-acryloyloxyethyl hydrogen phthalate of the formula III where R⁹ is benzoyl, R¹⁰ is bivalent —(OR)¹¹ᵣ—, where r=1 and R⁸ is H or methyl, preferably H optionally in admixture with 2-hydroxyethyl acrylate, 2-hydroxyethyl methacrylate or hydroxypropyl (meth)acrylate.

Equally preferred monomers as at least one further monomer in the composition may be selected from: (g) at least one di-, tri-, tetra- or multi-functional monomer which is in particular not a urethane (meth)acrylate.

Preferably, hydroxyethyl acrylate is used as the mono-functional monomer. Likewise, hydroxypropyl acrylate, hydroxyethyl methacrylate, hydroxypropyl methacrylate and/or hydroxyethyl acrylate can optionally be used as a mixture of at least two of the aforementioned monomers. Preferably, the at least one acrylic acid ester with additional carboxyl group is present in a mixture with hydroxyethyl acrylate, the acrylic acid ester with additional carboxyl group being present in an amount of from 95 to 99% by weight and the hydroxyethyl acrylate being present in an amount of from 1 to 5% by weight and the mixture has a total content of 100% by weight.

According to an alternative, compositions are preferred which comprise as a further component inorganic fillers selected from (f) inorganic fillers, inorganic oxides or inorganic mixed oxides, in particular oxides of zirconium and/or silicon and/or dental glasses, preferably silicon dioxide, zirconium dioxide or mixed oxides with silicon dioxide and a metal oxide, in particular metal dioxide and silicon dioxide. In the present case, metal oxides or metal dioxides are those that do not correspond to silicon dioxide, which is already mentioned as a special metal oxide. Mixed oxides with silicon dioxide and another metal oxide with primary particle sizes in the range of less than 100 nm, optionally present as agglomerates of 2 to 5 μm, are particularly preferred as metal oxides. These oxides or mixed oxides are preferably essentially X-ray amorphous. Aluminosilicate glasses, fluoroalumino silicate glasses and/or barium aluminum silicate can be used as dental glasses. The oxides can be selected from the aforementioned as well as from amorphous spherical fillers based on oxide or mixed oxide. The primary particles are the smallest particles of the oxides, which may be present as agglomerates.

The particle sizes of the inorganic fillers, such as the at least one inorganic oxide, mixed oxide or dental glass, for example comprising barium aluminum oxide, have an average particle diameter of d₅₀ less than 10 μm for the present applications, particularly preferably the fillers have a particle diameter of approximately 3 to 70 nm, in particular of 10 to 50 nm (nanometers), optionally the particles can be aggregated or agglomerated as particles with up to 10 μm. The primary particle sizes of the inorganic fillers, which can optionally be present as agglomerated and/or aggregated primary particles, have an average particle diameter of approximately 3 to 70 nm, in particular of 10 to 50 nm. Preferably, the inorganic oxides such as silicon dioxide have a primary particle size of 3 to 70 nm. The particle size can be determined in a dispersion in water by determining the particle size distribution by volume weight (Malvern, using laser diffraction, ISO13320:2009). Additionally, or alternatively, the particle size can be determined using SEM (scanning electron microscope). The advantage of the very small particle diameters, which may be aggregated and/or agglomerated, is that the light is essentially diffusely scattered by these particles during beam curing and thus leads to improved curing in the stereolithography process or DLP process.

A subject of the invention is a composition comprising monomers and optionally pre-polymers (oligomers) and/or polymers comprising at least one difunctional uretahn(meth)acrylate, at least one monofunctional acrylate with dipentanyl group and/or methacrylate with dipentanyl group, at least one acrylic acid ester with additional carboxy group, acrylic acid ester with at least one additional anhydride group of carboxy groups and/or derivative of the aforementioned acrylic acid esters and optionally at least one inorganic filler.

In order to meet high esthetic demands, compositions used in the dental sector for the fabrication of permanent restorations, such as working models, orthodontic models, drilling templates, temporary restorations and splints, must have a high degree of transparency. This transparency is usually achieved by optimally adjusting the refractive indices of the fillers and the polymer matrix. However, due to various physical and chemical boundary conditions, there are very strict limits to the choice of fillers and monomers.

A preferred filler content may be from 0 to 35% by weight with respect to the total composition.

Preferred fillers comprise silicon dioxide, zirconium dioxide, mixed oxides with silicon dioxide and/or at least one mixed oxide of zirconium oxide and silicon dioxide as well as mixtures comprising at least one of the inorganic oxides. Preferably, the filler content may be from 0.01 to 10% by weight of a mixed oxide and optionally additionally from 0 to 25% by weight of silicon dioxide. Preferred is a mixture, in particular with a filler content of 10 to 35% by weight with respect to the total composition, with a content of 1 to 10% by weight of the mixed oxide and 9 to 25% by weight of silicon dioxide.

Furthermore, it is preferred if the composition is not thixotropic. In addition, it is particularly preferred if the composition has a viscosity of less than 7500 m·Pas, in particular from 500 to 7000 m·Pas, preferably from 500 to less than 4000 m·Pas, preferably from 500 to 3000 m-Pas, particularly preferably from 500 to 1500 m·Pas. The viscosity is preferably measured according to DIN 1342-2; 2003-11 newtonian liquids or DIN 1342-3; 2003-11 non-newtonian liquids with a rheometer (Anton Par, physicist MCR 301, at a shear rate d(gamma)/dt=100/s 23° C.). The compositions according to the invention have no or preferably only a low thixotropy. According to a further embodiment, it is preferred that almost no viscosity changes occur in these over a longer storage period. Furthermore, the compositions exhibit very good reactivity when exposed to a laser or DLP projector.

With the composition according to the invention, workpieces or three-dimensional molded bodies can be printed with very good geometric precision/resolution. Furthermore, good color stability can be observed in the workpieces.

An object of the invention is a polymerized composition, preferably as a three-dimensional shaped body, in particular as a dental prosthetic part, orthopaedic appliance or dental preform, obtainable by irradiation of a polymerizable composition. Additional radiation curing on all sides is understood to mean, for example, post-curing in a 3D light oven.

Preferably, the polymerized compositions have a ratio of modulus of elasticity/flexural strength of greater than or equal to 25, preferably greater than or equal to 26, and in particular in combination with water absorption of less than or equal to 45 [g/mm³] according to ISO 10477.

Another object of the invention is a blank in the form of a three-dimensional moulded body of a polymerized composition, which has preferably additionally been radiation-cured on all sides, which is suitable for producing dental prosthetic parts, orthopaedic appliances, dental pre-forms, technical parts, tools, instruments, hoof repair parts or implants in medical prosthetics, the blank having a) a flexural strength of greater than or equal to 75 MPa, and/or b) a modulus of elasticity of greater than or equal to 75 MPa, tools, instruments, hoof repair parts or implants in medical prosthetics, wherein the blank has a) a flexural strength of greater than or equal to 75 MPa, and/or b) a modulus of elasticity of greater than or equal to 2600 MPa and/or c) a water absorption of less than 45 [g/mm³]. The blank can preferably be in the form of an artificial tooth or a bridge or as part of a bridge, which only needs to be polished for final individual adaptation to the patient, or the occlusal surface needs to be slightly reworked.

A further object of the invention is an article in the form of a three-dimensional moulded body of a polymerized composition in the form of dental prosthetic parts, orthopaedic appliances, dental pre-forms, technical parts, tools, instruments, hoof repair parts or implants in medical prosthetics, characterized in that the blank has a) a flexural strength of greater than or equal to 75 MPa according to DIN EN ISO 10477:2020 and/or b) a modulus of elasticity of greater than or equal to 2600 MPa according to DIN EN ISO 10477:2020 and/or c) has a water absorption of less than 45 [g/mm³].

A further object of the invention is the use of a composition for the manufacture of dental prosthetic parts, orthopaedic appliances, dental pre-forms or technical parts, tools, instruments, hoof repair parts or implants in medical prosthetics, in rapid prototyping or in rapid manufacturing, i.e. the manufacture of dental prosthetic parts or rapid tooling processes. Technical parts comprise all parts that are subject to mechanical stress, such as parts in the automotive sector, parts of machines, parts of engines, parts of fittings, parts of furniture, parts of consumer goods or parts of kitchen utensils.

The following processes—rapid prototyping or rapid manufacturing, a process for producing workpieces such as a dental prosthetic part, or rapid tooling, a process for producing tools—each comprise stereolithography processes and DLP processes. Optionally, after curing of the polymerizable composition in the aforementioned processes, post-treatment with UV, Vis or UV/Vis light can be carried out. Preferably, post-curing of the polymerized composition or the dental prosthetic parts, the orthopaedic appliances or dental pre-forms or blanks is carried out simultaneously from at least three sides, preferably from five to six sides, as is possible in a light oven.

The dental prosthetic parts comprise a denture base or parts thereof, e.g. as a reproduction of a gingiva or a part thereof, artificial teeth, dental arches with at least two to 16 artificial teeth connected interdentally in one piece, crowns, temporary crowns, full dentures, full crowns, splints for orthodontic corrections (similar to Invisalign), dental bridges, abutments, superstructures, dental bars, inlays, veneers, onlays, orthopaedic appliances such as occlusal splints, dental pre-forms for artificial teeth, drilling templates for implantology, mouthguards and/or implants.

Another object of the invention is a use as bone cement for cementing artificial joint prostheses, crowns, telescopes, veneers, dental bridges, prosthetic teeth, implants, implant parts, abutments, superstructures, orthodontic appliances. In addition to dental prosthetic parts, medical implants or use in the veterinary field, in particular as a hoof repair material, are also preferred.

Color pigments can also be added to the composition to adjust the color. Furthermore, red fibers can be added to the composition to imitate veins in the gingiva. In the polymerized composition, layer thicknesses in the range of 5 μm to 250 μm per curing layer can be achieved.

In an alternative, the mono-functional monomer may contain at least one of the monomers mentioned: Methyl methacrylate and optionally additionally ethyl methacrylate, propyl methacrylate, butyl methacrylate, n-hexyl methacrylate, 2-phenoxyethyl methacrylate, isobornyl methacrylate, isodecyl methacrylate, polypropylene glycol mono-methacrylate, tetrahydrofuryl methacrylate, polypropylene glycol mono-methacrylate, methyl acrylate, ethyl acrylate, propyl acrylate, butyl acrylate, n-hexyl acrylate, 2-phenoxyethyl acrylate, isobornyl acrylate, isodecyl acrylate, polypropylene glycol monoacrylate, tetrahydrofuryl acrylate, polypropylene glycol monoacrylate, benzyl acrylate, furfuryl acrylate or phenyl (meth)acrylate a mixture containing at least one of these (meth)acrylates and/or co-polymers comprising one or at least two of the aforementioned monomers.

Further, the composition may comprise monomers selected from tri-, tetra- or multi-functional monomer other than urethane (meth)acrylate, such as pentaerythritol tetraacrylate, trimethylolpropane tri(meth)acrylate and/or pentaerythritol tetra(meth)acrylate.

Suitable photoinitiators possible comprise benzoin alkyl ethers or esters, benzil monoketals, acyl phosphine oxides or aliphatic and aromatic 1,2-diketo compounds, such as 2,2-diethoxyacetophenone; 9,10-phenanthrenquinone, diacetyl, furil, anisil, 4,4′-dichlorobenzil and 4,4′-dialkoxybenzil or camphorquinone. The photoinitiators are preferably used together with a reducing agent. Examples of reducing agents are amines such as aliphatic or aromatic tertiary amines, for example N,N-dimethyl-p-toluidine or triethanolamine, cyanethylmethylaniline, triethylamine, N,N-dimethylaniline, N-methyldiphenylamine, N,N-dimethyl-sym.-xylidine, N,N-3,5-tetramethylaniline and 4-dimethyl aminobenzoic acid ethyl ester or organic phosphites. Common photoinitiator systems are, for example, camphorquinone plus ethyl 4-(N,N-dimethylamino)benzoate, 2-(ethylhexyl)-4-(N,N-dimethyl amino)benzoate or N,N-dimethylaminoethyl methacrylate.

2,4,6-Trimethylbenzoyldiphenylphosphine oxide, (bis-trimethylbenzoyl)-phenylphosphine oxide, ethyl (2,4,6-trimethylbenzoyl)phenylphosphinate (TPO-L), benzildimethylketal are particularly suitable as initiators for the polymerization initiated by UV light, 2-benzyl-2-dimethylamino-1-(4-morpholinophenyl)-butanone-1,4-benzoyl-4′ methyldiphenylsulphide, benzophenone, 1-hydroxy cyclohexyl phenylketone, camphorquinone, 2,2 diethoxyacetophenone, 2,4 diethyl thioxanthone, dimethylhydroxyacetophenone, 4,4-bis (diethylamino) benzophenone, 1-(9,9-dibutyl-9H-fluoren-2-yl)-2-methyl-2-morpholin-4-yl-propan-1-one, isopropylthioxanthone, 1-hydroxycyclohexylphenyl ketone and benzophenone, 4-methylbenzophenone and benzophenone, 2,4,6 trimethylbenzoyl-diphenylphosphine oxide, dimethylhydroxyacetophenone, methyl o-benzoyl benzoate, methyl benzoyl formate, 4-phenylbenzophenone, 2-methyl-1-(4-methylthiophenyl)-2-morpholinpropan-1-one and/or ethyl (2,4,6-trimethylbenzoyl) phenylphosphinate as well as mixtures comprising at least two of the photoinitiators. UV photoinitiators can be used alone or in combination with an initiator for visible light.

Suitable optional photoinitiators and/or initiator systems may comprise a) at least one free-radical photoinitiator, in particular at least one peroxide and/or azo compound, in particular LPO: dilauroyl peroxide, BPO: dibenzoyl peroxide, t-BPEH: tert.butyl per-2-ethylhexanoate, AIBN: 2,2′-azobis-(isobutyronitrile), DTBP: di-tert-butyl peroxide, or an alpha-hydroxyketone, camphorquinone, acylphosphine oxide. Optionally, stabilizers can also be added and optionally b) at least one co-initiator, such as an amine, usually a tert-amine, in particular at least one aromatic amine, such as N,N-dimethyl-p-toluidine, N,N-dihydroxyethyl-p-toluidine and/or p-dibenzylaminobenzoic acid diethyl ester.

Typical stabilizers comprise 2,6-di-tert-butyl-4-methylphenol (BHT), 2-hydroxy-4-methoxy benzophene and/or hydroquinone monomethyl ether (MEHQ).

### EXAMPLES

The invention is explained in more detail by the following examples, without limiting the invention to these embodiments.

Post-curing/post-tempering was carried out using a HiLite Power 3D laboratory light unit, among other things

### Method Description:

The specimens were printed with the Cara Print 4.0 (wavelengths: 385 nm and 405 nm) with a layer thickness of 50 μm with a suitable print data set according to the manufacturer's instructions and then post-cured for 2×5 minutes with HiLite Power 3D.

### Methods:

The measurements in Table 3 were carried out in accordance with DIN EN ISO 10477:2020 (23±2° C., at least 30% relative humidity unless otherwise specified. The DIN EN ISO 10477:2020 is referred to in full, i.e. the disclosure content is also disclosed. The specimens were manufactured from the printed specimens or flat bars, which were post-cured.

The flexural strength is determined according to DIN EN ISO 10477:2020, chapters 5.4 and 7.5, see FIG. 2 ISO 10477. Flexural strength specimens with a height h_{f}=2±0.1 mm and width b_{f}=2.0±0.1 mm, length l_{f}=25±2 mm are printed and exposed to light as described above. The specimens are tested after being stored in water at 37° C. for 24 hours. The test is performed using a Zwick universal testing machine with 0.4 N preload, 0.75 mm/min test speed. The corresponding modulus of elasticity (calculation of modulus of elasticity as secant) is determined from the measurement result of the flexural strength according to 7.5, ISO 10477 from the curve gradient (between 1 N and 7 N).

The three-media abrasion measurements are carried out according to the ACTA method (ISO/TS 14569-2, 2001, Dental materials—Guidance on testing of wear, Part 2, chapter 5). The test specimens with dimensions of 10×12 mm are printed, post-exposed (as described above) and fixed in the specimen wheel accordingly. After grinding (=creation of a uniform surface using diamond grinding wheels), abrasion is carried out in the same device (three-media abrasion machine DMA, SD Mechatronik) in a mixture of water and poppy seeds (110 g dried blue poppy seeds:205 g water) over 300,000 cycles. The antagonist and sample wheel run in opposite directions (contact pressure 20N, antagonist wheel at 240 rpm, sample wheel at 180 rpm). The poppy seed/water mixture is renewed after 150,000 cycles.

As the sample wheel is wider than the antagonist wheel, an unradiated area remains on each test specimen; this area is the reference height for evaluation. The evaluation (mean depth in μm and volume loss in mm³) is carried out with a non-contact surface laser scanner (OPM GmbH, resolution 100 P/mm, resolution z-axis 0.1 μm) on an area of 2×8 mm (of which approx. 1 mm on both sides is not abraded as reference height).

Water absorption and solubility are determined according to DIN EN ISO 10477:2020, chapters 5.6, 5.7 and 7.7. Round test specimens with a diameter of 15+/−1 mm and a thickness of 1+/−0.1 mm are printed, post-exposed (printing method as described above), polished to a high gloss according to an application situation and the diameter and thickness are measured individually to determine the volume (V). 5 Test specimens are conditioned in a desiccator over silica gel at 37° C. until constant weight (m1) is reached. This is followed by storage in water at 37° C. for 7 days. The weight after water storage is determined 1 min after removal and blotting with cellulose (m2). Re-drying takes place again in a desiccator at 37° C. until constant weight is reached (m3).

The water absorption results from (m2-m3)/V. The solubility is given by (m1-m3)/V. The maximum limit for water absorption according to the standard is <40 g/mμ³ and the limit for water solubility is <7.5 μg/m³.

### Design Examples

The mixture produced is used to print test specimens for the subsequent tests on a 3D precision printer with a wavelength of 405 nm (Cara Print 4.0). After the printing process, the test specimens are rinsed with isopropanol and subjected to a post-curing process. This is carried out by exposing both sides for 5 minutes or according to the manufacturer's instructions in a HiLite Power 3D, 200 W laboratory light lamp (Kulzer GmbH).

The properties of the mixture according to the invention as printed materials, in particular printed dental materials, are tested according to ISO 10477 (in water 37° C.) and according to the poppy abrasion described above.

It is assumed that the competitor Nextdent avoids the brittleness in the product C&B MFH by using a high proportion of HEMA (according to MSDS: 15-25%). As a result, the resulting printed blanks exhibit excessive water absorption and pronounced sedimentation was also observed. The composition of Nextdent is specified in the MSDS as follows: Urethane dimethacrylate 50 to 75% by weight, 2-hydroxymethacrylate <25% by weight, ethylene glycol dimethacrylate <10% by weight, ethoxylated bisphenol A dimethacrylate <10% by weight, diphenyl (2,4,6-trimethylbenzoyl) phosphine oxide 1 to 5% by weight.

TABLE 1
Examples

| | Comparison | | |
|---|---|---|---|
| | example 1 | | |
| | Nexdent | | |
| | readjusted | Example 1 | Example 2 |
| | [% by weight] | [% by weight] | [% by weight] |
| UDMA (urethane dimethacrylate) | 54.90995 | 54.90995 | 54.90995 |
| Dicyclopentanyl methyl acrylate | — | 24 | — |
| Acryloyloxy-ethyl hydrogen | — | — | 23.4 |
| phthalate | | | |
| Hydroxyethyl acrylate | — | — | 0.6 |
| 2-Hydroxyethyl methacrylate | 24 | — | — |
| (HEMA) | | | |
| Ethylene glycol dimethacrylate | 5 | 5 | 5 |
| Ethoxylated bisphenol A | 5 | 5 | 5 |
| dimethacrylate, n + m = 2 | | | |
| Diphenyl(2,4,6- | 1.0 | 1.0 | 1.0 |
| trimethylbenzoyl)phosphine oxide | | | |
| Silica | 10 | 10 | 10 |
| Pigments including titanium | 0.090 | 0.090 | 0.090 |
| dioxide | | | |
| BHT (2,6-di-tert-butyl-4- | 0.00005 | 0.00005 | 0.00005 |
| methylphenol) | | | |
| | 100.0 | 100.0 | 100.0 |

TABLE 2
Examples

| | Example 3 | Example 4 | Example 5 | Example 6 |
|---|---|---|---|---|
| | [% by weight] | [% by weight] | [% by weight] | [% by weight] |
| UDMA (urethane | 54.90995 | 59.90995 | 54.90995 | 54.90995 |
| dimethacrylate) | | | | |
| Dicyclopentanyl methyl | 19 | 19 | 24 | 19 |
| acrylate | | | | |
| Acryloyloxyethyl hydrogen | 4.9 | 4.9 | 4.9 | 9.75 |
| phthalate | | | | |
| Hydroxyethyl acrylate | 0.1 | 0.1 | 0.1 | 0.25 |
| 2-Hydroxyethyl | — | — | — | — |
| methacrylate (HEMA) | | | | |
| Ethylene glycol | 5 | — | — | — |
| dimethacrylate | | | | |
| Ethoxylated bisphenol A | 5 | 5 | 5 | 5 |
| dimethacrylate | | | | |
| Diphenyl(2,4,6- | 1.0 | 1.0 | 1.0 | 1.0 |
| trimethylbenzoyl)phosphine | | | | |
| oxide | | | | |
| Silica | 10 | 10 | 10 | 10 |
| Pigments including titanium | 0.090 | 0.090 | 0.090 | 0.090 |
| dioxide | | | | |
| BHT (2,6-di-tert-butyl-4- | 0.00005 | 0.00005 | 0.00005 | 0.00005 |
| methylphenol) | | | | |
| | 100.0 | 100.0 | 100.0 | 100.0 |

TABLE 3
Chemical-physical and mechanical properties (for averaged
results, the standard deviation is given in brackets)

| | Comparison | | | | | | | |
|---|---|---|---|---|---|---|---|---|
| | example 1 | | Example 1 | | Example 2 | | Example 3 | |
| Flexural strength, | 80.4 | (10.6) | 102.8 | (7.9) | 118.4 | (5.9) | 106.7 | (9.2) |
| [MPa] ISO 10477 | | | | | | | | |
| E-modulus, [MPa] | 2647 | (55) | 2968 | (87) | 3058 | (91) | 3166 | (47) |
| ISO 10477 | | | | | | | | |
| Viscosity [m · Pas] | 240 | | 679 | | 6580 | | 1037 | |
| Water absorption | 71.3 | (1.1) | 22.7 | (0.7) | 40.3 | (0.6) | 23.7 | (0.9) |
| [μg/mm³] ISO 10477 | | | | | | | | |
| Water solubility | 8.1 | (0.7) | 3.0 | (0.6) | 16.3 | (0.7) | 1.2 | (0.7) |
| [μg/mm³] ISO 10477 | | | | | | | | |
| Volume loss [mm³] | 0.408 | (0.018) | 0.395 | (0.020) | 0.412 | (0.019) | 0.354 | (0.013) |
| Poppy seed abrasion | | | | | | | | |
| Average depth [μm] | 40.1 | (1.9) | 39.4 | (1.1) | 39.9 | (1.8) | 34.4 | (1.1) |

Poppy seed abrasion

The monofunctional dicyclopentanyl methyl acrylate serves as a thinner and it is assumed that it can counteract the shrinkage of the radiation-cured composition. The compositions with a dicyclopentanyl methacrylate content also show very low water absorption and good modulus of elasticity as well as high flexural strength values. The acrylic acid ester with additional carboxy group is used as a monofunctional monomer and as a plasticizer and itself has a higher viscosity (according to specification >4000 m·Pas) compared to HEMA. The use of the acrylic acid ester counteracts sedimentation on the one hand and has a positive effect on the shrinkage of the composition, which is less pronounced than the effect of HEMA. The composition of Example 2 with the combination of UDMA and acrylic acid ester with additional carboxy group has the highest value for flexural strength without water storage and a high value for Young's modulus. Example 3 comprises the combination of UDMA, dicyclopentanyl methyl acrylate and acrylic acid ester with additional carboxy group and shows the highest modulus of elasticity without water storage and the lowest value for water solubility. Both examples 1 and 3 have the lowest water absorption and the lowest water solubility.

1. A polymerizable, radiation-curable composition comprising
(i) monomers and
(ii) at least one further component,
wherein
(i) the monomers comprise
(a) 30 to 70% by weight of at least one at least difunctional urethane (meth)acrylate,
(b) 10 to 40% by weight of at least one monofunctional acrylate with an alicyclic group and/or at least one monofunctional methacrylate with an alicyclic group,
(d) 5 to 40% by weight of at least one acrylic acid ester with additional carboxy group,
(e) from 1 to 5% by weight of at least one disubstituted 4,4′-di(oxabenzene)dialkylmethane of the formula I 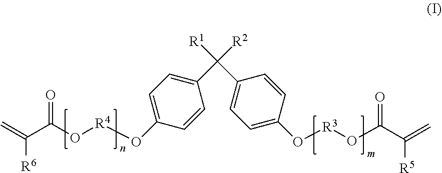
wherein R¹, R², R⁵ and R⁶ being each independently selected from H or C1 to C4 alkyl, and with R³ and R⁴ each bivalent C1 to C4 alkylene, with n=0 to 6 and m=0 to 6
(f) 0 to 35% by weight of inorganic fillers comprising inorganic oxides or inorganic mixed oxides and/or dental glasses,
and (ii) comprising at least one further component
(c) 0.01 to 10% by weight of at least one photoinitiator for the UV and/or Vis range or a photoinitiator system for the UV and/or Vis range,
the total composition being 100% by weight.

2. The composition according to claim 1, wherein it comprises
(f) 0 to 35% by weight of inorganic fillers comprising inorganic oxides or inorganic mixed oxides and/or dental glasses,
(g) 0 to 10% by weight, in particular 1 to 10% by weight of alkylene dimethacrylate and/or Alkylene diacrylate,
(h) optionally 0.1 to 5% by weight of hydroxyethyl acrylate,
the total composition being 100% by weight.

3. Composition according to claim 2, wherein it comprises as
(g) alkylene dimethacrylate ethylene glycol dimethacrylate.

4. A composition according to claim 1, wherein the composition is suitable for use in a generative printing process with layer-by-layer radiation-induced polymerization of the composition for the production of three-dimensional shaped bodies.

5. A composition according to claim 1, wherein the
(d) at least one acrylic acid ester with additional carboxy group is selected from an acrylic acid ester with additional carboxy group of formula II or III 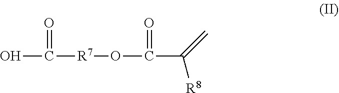
where R⁷ is in each case independently selected from bivalent C, H, O and optionally N-containing groups having 1 to 25 C-atoms, in particular bivalent aromatic esters, aromatic urethanes, alkylene esters, alkyl urethanes, aromatic ethers, alkyl ethers, and R⁸ is selected from H and 1 to 4 C-alkyl, preferably R⁷ is a bivalent aromatic ester, 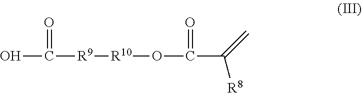
where R⁹ is independently selected from bivalent benzoyl, salicyloyl and derivatives thereof or —C—, R¹⁰ is bivalent —(OR)¹¹ᵣ— where R¹¹ is ethylene or propylene and where
r is 0 to 10, in particular 1 to 6, preferably r=1 or R¹⁰ is independently selected from bivalent alkylene and/or R⁸ is selected from H and 1 to 4 C-alkyl, preferably R⁸ is H, methyl or ethyl.

6. Composition according to claim 1, wherein
(b) the at least one monofunctional acrylate having an alicyclic group and/or at least one monofunctional methacrylate with alicyclic group comprising at least one monofunctional acrylate with a dicyclopentanyl group and/or at least one monofunctional methacrylate with a dicyclopentanyl group or mixtures containing these.

7. Composition according to claim 1, wherein
(b) the at least one monofunctional acrylate having an alicyclic group and/or at least one monofunctional methacrylate with alicyclic group comprising at least one monofunctional acrylate with a mono-valent alicyclic group and/or at least one monofunctional methacrylate with mono-valent alicyclic group.

8. Composition according to claim 2, wherein
(d) the at least one acrylic acid ester having an additional carboxy group comprising phthalic acid mono-[2-(methacryloyloxy)ethyl ester], 2-acryloyloxyethyl-hydrogen phthalate, polyether-functionalized acrylic acid esters with carboxy group and mixtures thereof.

9. A Composition according to claim 1, wherein
(a) the at least one di-functional urethane (meth)acrylate is selected from di-functional urethane (meth)acrylates having a bivalent alkylene group.

10. A Composition according to claim 7, wherein
(b) the at least one mono-functional acrylate having a mono-valent alicyclic group and/or at least one mono-functional methacrylate having a mono-valent alicyclic group comprising tricyclodecanealkanol methacrylate, tricyclodecanealkanol acrylate with alkanol having 1 to 10 C-atoms or mixtures containing at least one of these monomers.

11. Composition according to claim 1, wherein
(e) the at least one disubstituted 4,4′-di(oxabenzene)dialkylmethane of the formula I 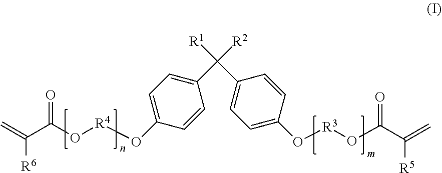
R¹ and R² each comprising methyl, and R⁵ and R⁶ being identical and selected from H, methyl and ethyl, in particular with R⁵ and R⁶ being identical and selected from H and methyl, and with R³ and R⁴ each independently bivalently ethylene or propylene with n=1 to 6, preferably n=2 to 4, and with m=1 to 6, preferably m=2 to 4, preferably with n=2 and m=2 or with n=4 and m=4 as well as mixtures thereof.

12. Composition according to claim 1, wherein composition comprises as further components inorganic fillers selected from, (f) inorganic fillers comprising inorganic oxides or inorganic mixed oxides and/or dental glasses.

13. Composition according to claim 1, wherein
the composition includes
(a) 40-60% by weight of at least one at least difunctional urethane (meth)acrylate,
(b) 15 to 30% by weight of at least one monofunctional acrylate with an alicyclic group and/or at least one monofunctional methacrylate containing an alicyclic group,
(c) 0.01 to 10% by weight of at least one photoinitiator for the UV and/or Vis range or a photoinitiator system for the UV and/or Vis range,
(d) 5 to 30% by weight of at least one acrylic acid ester with an additional carboxy group
(e) 1 to 15% by weight of at least one disubstituted 4,4′-di(oxabenzene) dialkylmethane of the formula I 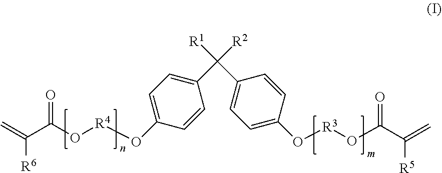
wherein R¹, R², R⁵ and R⁶ are each independently selected from H or C1 to C4
alkyl, and with R³ and R⁴ each bivalent C1 to C4 alkylene, with n=0 to 6 and
m=0 to 6.
(f) 0 to 35% by weight of inorganic fillers comprising inorganic oxides or inorganic mixed oxides and/or dental glasses, in particular silicon dioxide, zirconium dioxide, mixed oxides of a metal oxide and silicon dioxide, preferred fillers comprising silicon dioxide and/or mixed oxides of a metal dioxide and silicon dioxide, the total composition being 100% by weight.

14. A Composition according to claim 1, wherein the composition has a viscosity of less than 7500 μm·Pas, in particular from 500 to less than 4000 μm·Pas, preferably from 500 to 3000 μm·Pas.

15. Three-dimensional moulded body of a polymerized composition according to one claim 1 in the form of dental prosthetic parts, orthopaedic appliances, dental pre-forms, technical parts, tools, instruments, hoof repair parts or implants in medical prosthetics, wherein the moulded body a) has a flexural strength of greater than or equal to 75 MPa according to DIN EN ISO 10477:2020 and/or b) a modulus of elasticity of greater than or equal to 2600 MPa according to DIN EN ISO 10477:2020 and/or c) a water absorption of less than 45 [μg/mm³].

16. Blank in the form of a three-dimensional moulded body of a composition according to claim 1 for the production of dental prosthetic parts, orthopaedic appliances, dental pre-forms, technical parts, tools, instruments, hoof repair parts or implants in medical prosthetics, wherein the blank a) has a flexural strength of greater than or equal to 75 MPa according to DIN EN 10477:2020 and/or b) a modulus of elasticity of greater than or equal to 2600 MPa according to DIN EN ISO 10477:2020 and/or c) has a water absorption of less than 45 [μg/mm³].

17. Method of use a composition according to claim 1 for the manufacture of dental prosthetic parts, orthopaedic appliances, dental pre-forms, technical parts, tools, instruments, hoof repair parts, implants in medical prosthetics.

18. Composition according to claim 1 for use as a bone cement in the cementation of artificial joint prostheses, crowns, telescopes, veneers, dental bridges, denture teeth, implants, implant parts, abutments, superstructures, orthodontic appliances.

19. Composition according to claim 1, characterized in that the composition comprises as further components inorganic fillers selected from (f) inorganic fillers selected from silicon dioxide, zirconium dioxide, mixed oxides of a metal oxide with silicon dioxide and/or a mixed oxide of silicon dioxide and zirconium dioxide as well as mixtures comprising one of the said inorganic oxides.

20. Method of use of a composition according to claim 17, in a rapid prototyping or rapid manufacturing or rapid tooling process.
